(19) United States
(12) Patent Application Publication
(10) Pub. No.: US 2019/0274935 A1
(43) Pub. Date: Sep. 12, 2019

(54) Antiperspirant/deodorant Composition

(71) Applicant: Lonza Inc., Morristown, NJ (US)

(72) Inventors: Elias Pambou, Blackley (GB); Adam Griffiths, Blackley (GB); Juliana Aparecida dos Santos Leite, Barueri (BR)

(21) Appl. No.: 16/299,341

(22) Filed: Mar. 12, 2019

Related U.S. Application Data
(60) Provisional application No. 62/641,463, filed on Mar. 12, 2018.
(60) Provisional application No. 62/662,954, filed on Apr. 26, 2018.

(51) Int. Cl. A61K 8/26; A61K 8/34; A61K 8/37; A61K 8/06; A61Q 15/00

(52) U.S. Cl. CPC A61K 8/26; A61K 8/345; A61K 8/375; A61K 2800/30; A61Q 15/00; A61K 2800/524; A61K 8/062

(57) ABSTRACT

The invention provides an antiperspirant/deodorant composition having (a) 2 w/w %-30 w/w % of an antiperspirant/deodorant active; (b) 0.01 w/w %-20 w/w % of an emulsifier mixture, wherein the emulsifier mixture is one or more of a polyglycerol ester having an HLB value between 3 and 16, and (c) 30 w/w %-95 w/w % of an aqueous solvent, based on the total weight of the composition. The composition may be free of any emollients and may be incorporated into formulations for end-user body care products in the form of an aerosol, pump spray, liquid, roll-on, lotion, cream, gel, stick, solid, soft solid or antiperspirant/deodorant wipe.

The present application is based upon and claims priority to United States Provisional Application Ser. No. 62/641,463, filed on Mar. 12, 2018, and U.S. Provisional Application Ser. No. 62/662,954 Filed Apr. 26, 2018; which are incorporated herein by reference.

### FIELD OF INVENTION

The disclosure relates to an antiperspirant/deodorant composition. The end-user formulation of the composition can be used for body care products in the form of an aerosol, pump spray, liquid, roll-on, lotion, cream, gel, or stick antiperspirant/deodorant.

### BACKGROUND OF THE INVENTION

Current antiperspirant/deodorant compositions on the market often contain a humectant, viscosity modifier and emollient to assist with the aesthetic, spreading and viscosity properties of the formulation. The requirement of these added ingredients increases the cost of these antiperspirant/deodorant products to achieve a formulation that is accepted by the end user. Added cost in a product which is high volume, low margin makes it difficult to provide a cost effective and profitable product that is acceptable to the end user.

The opportunity of emitting the aforementioned components (such as humectant, viscosity modifier, emollients, and the like) would allow a manufacturer to increase available formulation space. This formulation space can be replaced with other components, or desirably water, which will significantly improve manufacturing costs. The advantage of such a composition means fewer components, easy formulation, effective stability, lower manufacturing cost and manageable transportability. The present disclosure provides an answer for the need for a simpler antiperspirant/deodorant formulation system, with limited ingredients and improved effectiveness.

There is a need in the art to provide a simpler antiperspirant/deodorant composition for end-user formulations, which has a reduced cost, but also provides all of the benefits of formulations containing additional ingredients such as humectants, viscosity modifiers and emollients, including, for example, rheological, aesthetic spreading and drying properties. This present invention meets this need in the art.

### SUMMARY OF THE INVENTION

The present invention provides a composition containing an antiperspirant/deodorant active for deodorant and antiperspirant products designed to control sweating or the body odor often associated with sweating. Such compositions are usually dissolved or dispersed in a carrier material which is in turn dispensed in end-user formulation for consumer body care products, usually to the underarm area.

In one aspect, the present invention provides an antiperspirant/deodorant formulation containing (a) an antiperspirant/deodorant active, (b) an emulsifier mixture of one or more polyglyceryl esters having an HLB value between 3 and 16 and (c) an aqueous solvent.

In accordance, the present invention describes an antiperspirant/deodorant composition containing: (a) 2 w/w %-30 w/w % of an antiperspirant/deodorant active; (b) 0.01 w/w %-20 w/w % of an emulsifier, wherein the emulsifier is one or more of a polyglycerol ester having an HLB value between 3 and 16, and (c) 30 w/w %-95 w/w % of an aqueous solvent, based on the total weight of the composition.

In one particular embodiment, the invention provides an antiperspirant/deodorant composition containing an emollient, whereby the emollient is present in an amount of 1 w/w %-10 w/w %; and in another embodiment the emollient is present in an amount less than 1 w/w %, based on the total weight of the composition.

In a further embodiment, the antiperspirant/deodorant composition is completely free of an emollient.

In another embodiment, the antiperspirant/deodorant composition contains (a) an antiperspirant/deodorant active is present in an amount of 5 w/w %-20 w/w %; (b) an emulsifier, wherein the emulsifier is one or more of a polyglycerol ester is present in an amount of 1 w/w %-7 w/w %; and c) an aqueous solvent present in an amount of 60 w/w %-95 w/w %, based on the total weight of the composition.

In one embodiment, the antiperspirant/deodorant active is an aluminum salt, a zinc salt, a zirconium salt, a deodorant active or mixtures thereof. In one particular embodiment, the antiperspirant/deodorant active is an aluminum salt, specifically aluminum chlorohydrate.

In one embodiment, the emulsifier comprises a polyglycerol ester, having an HLB value between 3 and 16, derived from (a) a polyglycerol component built up from 2 molecules to 12 molecules of glycerol, based on an average, and (b) a fatty acid comprising caprylic acid, capric acid, lauric acid, myristic acid, palmitic acid, stearic acid, arachidic acid, behenic acid, lignoceric acid, cerotic acid, oleic acid, decaoleic acid or mixtures thereof.

In another embodiment, the emulsifier is one or more of a polyglycerol esters having a HLB value between 3 and 16 comprising polyglyceryl-10 decaoleate, polyglyceryl-3 monostearate, polyglyceryl-6 distearate, polyglyceryl-10 stearate, polyglyceryl-10 oleate, polyglyceryl-10 dipalmitate, polyglyceryl-10 palmitate, polyglyceryl-10 caprylate/caprate; or mixtures thereof.

In one embodiment, the aqueous solvent comprises water, aqueous alcohols, ammonia water, acid solutions, salt solutions, water-miscible organic solvents or mixtures thereof. In one particular embodiment, the aqueous solvent is water.

In yet another embodiment, the addition of a humectant to the antiperspirant/deodorant composition is included, whereby the humectant is present in an amount of 0.01 w/w %-10 w/w %, based on the total weight of the composition.

In one embodiment wherein the antiperspirant/deodorant composition contains a humectant, wherein the humectant comprises a polyol. In a particular embodiment, the humectant comprises glycerol.

In one embodiment, an additional emulsifier is included, containing one or more of an ethoxylated fatty alcohol and ethoxylated fatty acids, ethoxylated sorbitan esters, in particular fatty esters of ethoxylated sorbitan or combinations thereof. In a further embodiment, the additional emulsifier is an ethoxylated fatty alcohol.

In one embodiment, the invention further comprises at least one of a viscosity modifier, fragrance, co-surfactant, cosmetic additive, biocidal agent, chelating agent, antioxidant, essential oil, preservative, vitamin, moisturizer, combinations thereof, and the like.

In one embodiment, the antiperspirant/deodorant composition further comprises a preservative, wherein the preservative is present in an amount of 0.01 w/w %-10 w/w %, based on the total weight of the composition.

In another embodiment, the preservative comprises a blend of gluconolactone, sodium benzoate and calcium gluconate.

In another aspect, the present invention provides an antiperspirant/deodorant composition in the form of an aerosol, pump spray, liquid, roll-on, lotion, cream, gel, stick, solid, soft-solid, or deodorant wipes.

In another embodiment, the present invention provides an antiperspirant/deodorant composition comprising: a) 2 w/w %-30 w/w % of an antiperspirant/deodorant active; (b) 0.01 w/w %-20 w/w % of an emulsifier, wherein the emulsifier contains one or more polyglycerol esters having an HLB value between 3 and 16, and (c) 30 w/w %-95 w/w % of an aqueous solvent, based on the total weight of the composition; and wherein the composition is an oil-in water emulsion for an aerosol, pump spray, liquid, roll-on, lotion, cream, gel, stick, or deodorant wipe.

These and other aspects will become apparent when reading the detailed description of the invention.

### DETAILED DESCRIPTION

It has now been surprisingly found that a stable, simpler antiperspirant/deodorant composition may be formed from the combination of an antiperspirant/deodorant active, one or more polyglyceryl esters, and an aqueous solvent. The aesthetic properties and rheological behavior of polyglycerol esters means that additional components are not required, thereby reducing the manufacturing cost, time and number of ingredients used in the end-user formulations. Other additives conventionally used in antiperspirant/deodorant composition may be added to the composition of the present invention for their know and intended purpose, provided that the optional conventional ingredients, do not adversely affect the properties within the antiperspirant/deodorant composition within the present invention.

Polyglyceryl esters are used as multifunctional emulsifiers to create antiperspirant formulations in the form of either an oil-in-water (o/w) emulsion system or a structured water system. Oil-in-water systems can defined as systems where water, or a water-soluble solution, makes up the continuous phase and oil the dispersed phase; in that oil droplets (the dispersed phase) can be stabilized (or emulsified) by an emulsifier in such a way that they are dispersed in a water-based solution (the continuous phase). The presence of an emulsifier is required to prevent the interaction and coalescence of the oil droplets which could eventually lead to separation of the oil and water phase, and therefore an unstable product. Generally, for o/w emulsions the water content (continuous phase) is much greater than 50 w/w %.

The present invention encompasses an antiperspirant/deodorant composition containing an antiperspirant/deodorant active, one or more emulsifiers and an aqueous solvent. One unique aspect of the invention is that the composition is free of any emollients; and the composition is very stable over an extended period of time.

The addition of an emollient, humectants and the like, although not necessary, may be added if desired, without disturbing the properties within the antiperspirant/deodorant composition. A further advantage of the antiperspirant/deodorant composition is that the composition may be formulated for end-use body care products in the form of an aerosol, pump spray, liquid, roll-on, lotion, cream, gel, powder, stick, solid, soft solid, and/or antiperspirant/deodorant wipes, and the like.

The term “HLB”, herein, refers to “hydrophilic-lipophilic balance”. The HLB value expresses the water and oil solubility of, in particular, non-ionic, emulsifiers (i.e. it expresses the lipophilic and hydrophilic properties of an emulsifier, which are determined by the different parts of the emulsifier molecules). The higher the HLB of an emulsifier, the more hydrophilic and water soluble it is. According to the Griffiths method, HLB is calculated via: HLB=(molecular weight of hydrophilic head-group/total molecular weight)*20.

The term “free”, herein, refers to a composition containing no amount of a particular substance. The term “free” should also be understood as being completely free of a particular substance.

The term “antiperspirant/deodorant active” herein, refers to any substance that has antiperspirant activities and/or deodorant activities either used alone or in combination.

The term “humectants” herein, refers to any substance capable of providing moisturizing and/or conditioning properties; deliver hydration and/or nutrients; substance that promotes retention of moisture; substances that actually bonds with water molecules to increase the water content to the composition, and the like. Humectants typically draw water to the skin from two different places; they can draw water from a humid environment, and they enhance water absorption from the outer layer of skin.

The term “structured water system” herein, refers to the presence of an emulsifier in solutions causes an increase of viscosity. The increase in viscosity is caused the self-assembly and arrangement (packing) of the emulsifier chemistry in such a way that ordered structures are formed in bulk liquid, which trap the liquid and prevent it from flowing.

In accordance with the present invention, the antiperspirant/deodorant composition contains (a) 2 w/w %-30 w/w % of an antiperspirant/deodorant active; (b) 0.01 w/w %-20 w/w % of an emulsifier, wherein the emulsifier mixture is one or more of a polyglycerol ester having an HLB value between 3 and 16, and (c) 30 w/w %-95 w/w % of an aqueous solvent, based on the total weight of the composition.

Suitably, the antiperspirant/deodorant composition contains (a) the antiperspirant/deodorant active present in an amount of 5 w/w %-20 w/w %; (b) the emulsifier is one or more of polyglycerol esters present in an amount of 1 w/w %-7 w/w %; and c) an aqueous solvent present in an amount of 60 w/w %-95 w/w %, based on the total weight of the composition.

If desired, an emollient may be used, and if used in high levels, the antiperspirant/deodorant composition of the present invention may also be in the form of an emulsion specifically whereby the emulsion is an oil-in-water emulsion.

Typically, antiperspirant/deodorant composition contains antiperspirant/deodorant active present in an amount of 2 w/w %-30 w/w %; Examples of such actives include, but are not limited to an aluminum salt, a zinc salt, a zirconium salt, deodorant actives, mixtures thereof, and the like. Suitable examples include, but are not limited to deodorant actives such as, triclosan, triclocarban, usnic acid salts, zinc phenolsulfonate, P-chloro-D-alanine, D-cycloserine, aminooxyacetic acid, cyclodextrin, sodium bicarbonate, fragrances, combinations thereof, derivatives thereof and the like.

Suitably, antiperspirant/deodorant composition contains antiperspirant/deodorant active present in an amount of 5 w/w %-20 w/w %. Examples of such actives include, but are not limited to, aluminum salt, a zinc salt, a zirconium salt, mixtures thereof, and the like. Most desirably, the antiperspirant/deodorant active is an aluminum salt, specifically aluminum chlorohydrate.

Typically, examples of aluminum salts include, but are not limited to, aluminum chlorohydrate, aluminum chloride, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrate polyethylene glycol, aluminum sesquichlorohydrate propylene glycol, ammonium silver zinc aluminum silicate, calcium aluminum borosilicate, cobalt aluminum oxide, magnesium/aluminum/hydroxide/carbonate, magnesium aluminum silicate, magnesium/aluminum/zinc/hydroxide/carbonate, potassium alum, potassium aluminum polyacrylate, silver magnesium aluminum phosphate, Alums, sodium alum, sodium aluminate, sodium aluminum chlorohydroxylactate, sodium/aluminum hydroxide/oxalate/sulfate, sodium/aluminum/iron hydroxide/oxalate/sulfate, sodium/aluminum/iron/sulfate/citrate/hydroxide, sodium silver aluminum silicate, sodium/aluminum/iron/sulfate/oxalate/hydroxide, sodium aluminum lactate, sodium/aluminum/iron/sulfate/tartarate/hydroxide, sodium potassium aluminum silicate, sodium phosphorus/zinc/calcium/silicon/aluminum/silver oxides, sodium silicoaluminate, tromethamine magnesium aluminum silicate, other complexes or derivatives thereof, and the like.

Examples of zirconium salts include, but are not limited to, aluminum-zirconium octachlorohydrate, aluminum-zirconium octachlorohydrex gly, aluminum-zirconium pentachlorohydrate, aluminum-zirconium pentachlorohydrex gly, aluminum-zirconium tetrachlorohydrate, aluminum-zirconium tetrachlorohydrex gly, aluminum-zirconium trichlorohydrate, aluminum-zirconium trichlorohydrex gly, aluminum zirconium tetrachlorohydrex peg, aluminum zirconium tetrachlorohydrex peg, aluminum zirconium pentachlorohydrate, aluminum zirconium pentachlorohydrex gly, aluminum zirconium octachlorohydrate, aluminum zirconium octachlorohydrex gly combinations thereof, complexes thereof, or derivatives thereof, and the like.

Examples of zinc salts include, but are not limited to, zinc phenolsulphonate, zinc salts of acetic acid, lactic acid, oxalic acid, succinic acid, fumaric acid, maleic acid, ricinoleic acid and/or citric acid, combinations thereof, complexes thereof, or derivatives thereof, and the like.

If desired, the composition further comprises an emulsifier present in an amount of 0.01 w/w %-20 w/w %; whereby the emulsifier includes one or more of a polyglycerol ester, having an HLB value between 3 and 16. Suitably, the emulsifier is present in an amount of 1 w/w %-8 w/w %, whereby the emulsifier includes one or more of a polyglycerol ester, having an HLB value between 3 and 16.

Typically, the HLB value is between 3 and 16; suitably, the HLB value is at least 5 or higher; between 5 and 16; and desirably the HLB value is at least 8 or higher; between 8 and 16.

Examples of polyglycerol esters include, but are not limited to saturated, unsaturated, natural or synthetic fatty acids, and the like. For example, saturated fatty acids include caprylic acid, capric acid, lauric acid, myristic acid, palmitic acid, stearic acid, arachidic acid, behenic acid, lignoceric acid, cerotic acid, combinations thereof, derivatives thereof, and the like. Furthermore, the polyglycerol esters are derived from (a) a polyglycerol component built up of 2 to 12 molecules of glycerol, based on an average, and (b) a fatty acid comprising caprylic acid, capric acid, lauric acid, myristic acid, palmitic acid, stearic acid, arachidic acid, behenic acid, lignoceric acid, cerotic acid, oleic acid, decaoleic acid, mixtures thereof and the like.

Examples of polyglycerol esters, having an HLB value between 3 and 16, include, but are not limited to, polyglyceryl monodecaoleate such as polyglyceryl-10 decaoleate; polyglyceryl monooleate such as polyglyceryl-2-monooleate, polyglyceryl-3 monooleate, polyglyceryl-4 monooleate, polyglyceryl-6 monooleate, or polyglyceryl-10 monooleate; polyglyceryl dioleate such as polyglyceryl-2 dioleate, polyglyceryl-3 dioleate, polyglyceryl-5 dioleate, polyglyceryl-6 dioleate or polyglyceryl-10 dioleate; polyglyceryl trioleate such as polyglyceryl-5 trioleate or polyglyceryl-10 trioleate; polyglyceryl tetraoleate such as polyglyceryl-2 tetraoleate, polyglyceryl-6 tetraoleate, or polyglyceryl-10 tetraoleate; polyglyceryl pentaoleate such as polyglyceryl-4 pentaoleate, polyglyceryl-6 pentaoleate, or polyglyceryl-10 pentaoleate; polyglyceryl heptaoleate such as polyglyceryl-6 heptaoleate, polyglyceryl-10 heptaoleate; polyglyceryl monostearate such as polyglyceryl-2 monostearate, polyglyceryl-3 monostearate, polyglyceryl-4 monostearate, polyglyceryl-5 monostearate, polyglyceryl-6 monostearate or polyglyceryl-10 monostearate; polyglyceryl distearate such as polyglyceryl-2 distearate, polyglyceryl-3 distearate, polyglyceryl-4 distearate, polyglyceryl-6 distearate, or polyglyceryl-10 distearate; polyglyceryl tristearate such as polyglyceryl-4 tristearate, polyglyceryl-5 tristearate, polyglyceryl-6 tristearate, or polyglyceryl-10 tristearate; polyglyceryl tetrastearate such as polyglyceryl-2 tetrastearate; polyglyceryl pentastearate such as polyglyceryl-4 pentastearate, polyglyceryl-6 pentastearate, or polyglyceryl-10 pentastearate; polyglyceryl heptastearate such as polyglyceryl-10 heptastearate; polyglyceryl isostearate such as polyglyceryl-2 isostearate, polyglyceryl-3 isostearate, polyglyceryl-4 isostearate, polyglyceryl-6 isostearate, or polyglyceryl-10 isostearate; polyglyceryl diisostearate such as polyglyceryl-2 diisostearate. polyglyceryl-3 diisostearate, polyglyceryl-4 diisostearate, polyglyceryl-6 diisostearate, polyglyceryl-10 diisostearate, or polyglyceryl-15 diisostearate; polyglyceryl triisostearate such as polyglyceryl-2 triisostearate, polyglyceryl-3 triisostearate, polyglyceryl-5 triisostearate, polyglyceryl-10 triisostearate; polyglyceryl tetraisostearate such as polyglyceryl-2 tetraisostearate; polyglyceryl caprylate such as polyglyceryl-2 caprylate, polyglyceryl-3 caprylate, polyglyceryl-4 caprylate, polyglyceryl-6 caprylate, or polyglyceryl-10 caprylate; polyglyceryl dicaprylate such as polyglyceryl-5 dicaprylate; polyglyceryl sesquicaprylate such as polyglyceryl-2 sesquicapyrlate; polyglyceryl octacaprylate such as polyglyceryl-6 octacaprylate; polyglyceryl caprate such as polyglyceryl-2 caprate, polyglyceryl-3 caprate, polyglyceryl-4 caprate, polyglyceryl-5 caprate, polyglyceryl-6 caprate, polyglyceryl-10 caprate, polyglyceryl dicaprate such as polyglyceryl-3 dicaprate or polyglyceryl-6 dicaprate; polyglyceryl caprylate/caprate such as polyglyceryl-4 capyrlateicaprate, polyglyceryl-6 caprylate/caprate, or polyglyceryl-10 caprylate/caprate; polyglyceryl palmitate such as polyglyceryl-2 palmitate, polyglyceryl-3 palmitate, polyglyceryl-6 palmitate or polyglyceryl-10 palmitate; polyglyceryl dipalmitate such as polyglyceryl-6 dipalmitate or polyglyceryl-10 dipalmitate; polyglyceryl tetrabehenate such as polyglyceryl-6 tetrabehenate; polyglyceryl myristate such as polyglyceryl-6 myristate or polyglyceryl-10 myristate; polyglyceryl rincinoleate such polyglyceryl-6 polyricinoleate or polyglyceryl-10 ricinoleate; or mixtures thereof, other complexes or derivatives thereof, and the like.

Suitably, the polyglycerol ester comprises one or more of a polyglyceryl-10 decaoleate, polyglyceryl-3 monostearate, polyglyceryl-6 distearate, polyglyceryl-10 stearate; polyglyceryl-10 oleate, polyglyceryl-10 dipalmitate, polyglyceryl-10-palmitate, polyglyceryl-10-laurate, polyglyceryl-10 caprylate/caprate.

Typically, the antiperspirant/deodorant composition contains 30 w/w %-95 w/w % of an aqueous solvent; suitably the composition may contain 50 w/w %-95 w/w % of an aqueous solvent; and desirably, the antiperspirant/deodorant composition contains 60 w/w %-95 w/w % of an aqueous solvent.

Exemplary aqueous solvents include, but is not limited to, water, aqueous alcohols, ammonia water, acid solutions, base solutions, salt solutions; water-miscible organic solvents, mixtures thereof, and the like. Suitably, the aqueous solvent is water.

In addition to components (a), (b) and (c) described above, the antiperspirant/deodorant composition may further contain an emollient, wherein the emollient is present in an amount of 1 w/w %-10 w/w %; or in an amount less than 1 w/w %, based on the total weight of the composition.

Furthermore, the antiperspirant/deodorant composition may also be free of an emollient, meaning that the composition is completely free emollient of a particular substance other than true amounts.

If an emollient is desired, as an additional component of the antiperspirant/deodorant composition, an emollient such as waxes, oils, mixtures thereof, and the like may be used.

The term “waxes” herein, refer to naturally-occurring or synthetic waxes, of which typical examples include stearyl alcohol, hydrocarbon waxes, waxes of plant or animal origin, their synthetic analogues or derivatives, or silicone waxes, Waxes are widely available, and by suitable selection of the waxes themselves and their concentrations in the formulation can effectively obtain either a soft solid or a firm solid. Conventionally, waxes are applied to a variety of materials and mixtures which have similar physical properties; namely that: they are solid at about 30° C. to about 40° C.; they melt to a mobile liquid at a temperature above 30° C., and below 95° C.; generally in a temperature range of 40° C., to 90° C.; and they are water-insoluble and remain water-immiscible when heated above their melting point.

A variety of waxes may be suitable, such as silicone polymers, hydrocarbons, linear fatty alcohols, esters of fatty acids or glyceride derivatives, mixtures thereof, or complexes thereof; and the like.

Examples of waxes that may be used, for soft solid/stick antiperspirant/deodorant applications, include but are not limited to, castor wax, beeswax, carnauba and candelilla waxes, which are of vegetable origin and mineral waxes from fossil remains other than petroleum. Montan wax, which is an example of mineral wax, includes non-glyceride esters of carboxylic acids, hydrocarbons and other constituents. Other naturally available waxes include spermeceti wax, ozokerite, ceresin, baysberry, synthetic waxes, paraffin wax, silicone waxes or mixtures thereof, and the like.

The term “oils” herein, refer to an organic compound which at about 20° C. is both liquid and water-insoluble. In the context of the invention, insolubility in water is understood to be a solubility of less than 10% by weight at about 20° C. Typically, the solubility of less than 1 w/w %, more particularly less than 0.1 w/w %, in particular less than 0.01 w/w %.

Examples of oil emollients include, but are not limited to, ester oils, an ether oil or a mixture thereof, and the like. As used herein, the term “ester oil” refers to an oil as above-defined, which comprises at least one ester group. This means that also esters of carbonic acids are ester oils according to the invention. Further, as used herein, the term “ether oil” means an oil as above-defined, which comprises at least one ether group.

Typical examples of oils include, but are not limited to, glycerides such as e.g., triglycerides, hydrocarbons such as e.g., petrolatum; silicone oils such as e.g., dimethicone, dialkyl ethers, alkyl esters, dialkyl carbonates and natural oils such as vegetable oils. In dialkyl ethers, alkyl esters and dialkyl carbonates such as dicaprylyl carbonate, ethylhexyl carbonate, dihexyl carbonate; the alkyl groups may be straight or branched and independently from each other typically have C₂-C₁₆ atoms. Suitably, at least one of said alkyl groups has at least 6 carbon atoms, typically at least 8 carbon atoms. Optionally the oil may be an oil that has further functions such as an oil soluble sunscreen. Mixtures of oil components may also be used.

Suitably, examples of oils include, but are not limited to, grape seed oil (Vitis vinifera), sunflower seed oil (Helianthus annuus), olive oil, canola oil, safflower oil, hemp seed oil, marula oil, CBD oil, argan oil, other essential oils, combinations thereof and the like. Typically such oils are present in an amount of 0.01 w/w %-10 w/w %, based on the weight of the total composition.

If the composition contains an emollient, the emollient comprises a dialkyl ether is dicaprylyl ether, or an alkyl ester, C₁₂-C₁₅ alkyl benzoates, Typically, the emollient comprises a dialkyl carbonates are dicaprylyl carbonate, ethylhexyl carbonate, dihexyl carbonate and a mixture thereof, wherein dicaprylyl carbonate is suitably used.

The composition of the present invention may further comprise, an additional emulsifier, wherein the additional emulsifier is one or more of an ethoxylated fatty alcohols and ethoxylated fatty acids, exhoxylated sorbitan esters, in particular fatty esters of ethoxylated sorbitan, combinations thereof, and the like.

If desired, the additional emulsifier comprises an ethoxylated fatty alcohol having a C₄-C₃₆ atom chain, generally with a chain length of the fatty alcohol is about C₆-C₂₈ atoms; and whereby the chain length of the fatty alcohol is specifically about C₁₂-C₂₂ atoms.

Examples of such fatty alcohols include, but are not limited to, cetyl alcohol, stearyl alcohol, lauryl alcohol, myristyl alcohol, behenyl alcohol, mixtures thereof, and the like.

In one aspect, the ratio of the polyglycerol ester to the additional emulsifier(s) may be present in a weight ratio of about 1:10 to 10:1; in another aspect, the weight ratio of the polyglyceryl esters to the additional emulsifier(s) may be from about 1:5 to about 10:1; and in a further aspect, the weight ratio of the polyglycerol ester to additional emulsifier may be from about 1:1 to about 5:1.

Suitably, the antiperspirant/deodorant composition may further contain a humectant; whereby the humectant is present in an amount of 0.01 w/w %-10 w/w %, based on the total weight of the composition.

Typically, humectants that may be added are those that do not cloud or discolor the concentrate, whether natural or synthetic. Examples of humectants include, but are not limited to, molecule with several hydrophilic groups; silicones, amino acids, sugars, proteins, pyrrolidone carboxylic acid, lactic acid, hyaluronic acid, their salts thereof; lactitol, urea and urea derivatives (e.g., hydroxyethyl urea, uric acid), glucosamine, creatinine, cleavage products of collagen, chitosan and chitosan salts/derivatives (e.g., chitosan ascorbate), polyols, glycols, mixtures thereof, derivatives thereof, and the like. Particularly, the humectant comprises a polyol.

The term “polyols” herein, refer to organic compounds that comprise at least two hydroxyl groups. Generally, the polyol is a straight or branched hydrocarbon chain having at least two hydroxyl groups, wherein the hydrocarbon chain may optionally comprise one or several heteroatoms (e.g., oxygen or sulfur).

Examples of polyols include, but are not limited to, glycerol (glycerin), polyglycerols, such as diglycerol, triglycerol or polyglycerin-6, ethylene glycol, propylene glycol, butylene glycol, erythritol, 1,2,6-hexanetriol, polyethylene glycols, sugars and sugar derivatives (e.g., fructose, glucose, maltose, maltitol, mannitol, inositol, sorbitol, sorbitol silanediol, sucrose, trehalose, xylose, xylitol, glucuronic acid and salts thereof), ethoxylated sorbitols (Sorbeth-6, Sorbeth-20, Sorbeth-30, Sorbeth-40), honey and hydrogenated honey (as used herein, “hydrogenated honey” refers to the end product of controlled hydrogenation of honey), hydrogenated starch hydrolyzates and mixtures of hydrogenated wheat protein and PEG-20-acetate copolymer, mixtures thereof, and the like.

If a humectant is desired in the antiperspirant/deodorant composition, the humectant may be a polyol, in particular propylene glycol, glycerol, diglycerols and/or triglycerols. When a humectant is present, glycerol is generally used.

Optionally, the composition may further comprise a variety of additional components, including at least one of a viscosity modifier, fragrance, co-surfactant, cosmetic additive, biocidal agent, deodorant active, chelating agent, preservative, antioxidant, essential oil, vitamin, moisturizer, mixtures thereof, and the like. Any additional component may be added the antiperspirant/deodorant composition without disturbing the properties and end-user formulation for which the antiperspirant/deodorant composition may be used.

Examples of chelating agents include, but are not limited to, ethylenediaminetetraacetic acid (EDTA) and salts thereof (such as tetrasodium EDTA, diammonium EDTA, dipotassium EDTA, disodium EDTA, tetrahydroxypropyl ethylenediamine, HEDTA, tripotassium EDTA, trisodium HEDTA), dimercaprol, alanine diacetic acid, aminotrimethylene phosphonic acid, caprylhydroxamic acid, cyclohexanediamine tetraacetic acid, decapeptide-7, di-TEA-cocamide diacetate, dicarboxypyridine, oxalates (such as diisopropyl oxalate, dipotassium oxalate), galactaric acid, disodium hydroxyethyliminodiacetate, ilomastat, oxalic acid, pentasodium aminotrimethylene phosphonate, pentetic acid and salts thereof, phytic acid and salts thereof, potassium EDTMP, sodium EDTMP, pyricarbate, salicyloylhydroxamic acid, mixtures thereof, and the like.

Suitable preservatives include, but are not limited to, organic acids or salts thereof; such as for example, benzoic acid, salts of benzoic acid, salicylic acid, salts of salicylic acid, sorbic acid, salts of sorbic acid, dehydroacetic acid, salts of dehydroacetic acid; lactic acid or salts thereof, organic alcohols such as, for example, phenoxyethanol, benzyl alcohol, glycols e.g, caprylyl glycol and ethanol; quaternary ingredients such as, for example, Quaternium-15, or benzethonium chloride; isothiazolones, chlorhexidine, imidazolidinyl urea, diazolidinyl urea, sodium hydroxymethylglycinate, ethylhexylglycerol, hydantoins, iodopropynyl butylcarbamate, polyaminopropyl biguanide, parabens, natural extracts, botanical extracts, essential oils, or actives present on the Annex V preservative list, mixtures thereof, and the like.

Examples of commercially available preservatives, include but are not limited to, those available from Lonza, Allendale, N.J. such as the Cosmocil®, Georgard®, Glycacil®, Glydant®, Lonzagard®, Lonzaserve®, Mikrokill® and Spectradyne® series of preservatives may be used. Suitably, useful preservative is Geogard® Ultra, which is a blend of gluconolactone, sodium benzoate and calcium gluconate.

The preservative is generally added in an amount of 0.01 w/w % to about 10 w/w %, based on the total weight of the composition. In a further embodiment the preservative system is present in an amount of 0.1 w/w % to about 7 w/w %, based on the total weight of the composition. The preservative may be a single preservative or a combination of one or more preservatives.

In one embodiment, the antiperspirant/deodorant composition having a Brookfield viscosity value from about 0.1 to 50 Pas at about 25° C. and suitably a value from about 0.5 to 20 Pas at about 25° C. Typically a roll-on antiperspirant solution will have a Brookfield viscosity value from about 0.5 to 20 Pas at about 25° C.

Additionally, stability testing of one embodiment of the antiperspirant/deodorant composition was performed at about 25° C. to about 45° C. respectively for 1 month; with results showing complete stability of the composition after the 1 month period. Specifically, the stability testing was performed at 25° C., 37.5° C., and 45° C. respectively, for 1 month.

Another aspect of the present invention is the impregnated end-user formulation of the antiperspirant/deodorant composition, into an end-use body care product for consumers, in the form of an aerosol, pump spray, liquid, roll-on, lotion, cream, gel, sticks, solid, soft solid, deodorant wipes, other antiperspirant/deodorant based products, and the like. The amount of the antiperspirant/deodorant composition that may be used in end-user formulation can range of 1 w/w %-100 w/w %, based on the weight of the total formulation; suitably in a range of 1 w/w %-75 w/w % and desirably in a range of 1 w/w %-50 w/w %.

Additionally, if desired, a formulation of an end-user soft solid, stick, cream or roll-on, wipe, and/or lotion antiperspirant or deodorant may be formed using the composition of the present invention whereby component (c) of the present composition may be substituted with a wax based substance. For example, the end-user antiperspirant or deodorant composition may contain (a) 2 w/w %-30 w/w % of an antiperspirant/deodorant active; (b) 0.01 w/w %-20 w/w % of an emulsifier, wherein the emulsifier is 1 or more of a polyglycerol ester having an HLB value between 3 and 16, and (c) 30 w/w %-95 w/w % of a wax, based on the total weight of the composition.

Furthermore, one aspect of the present invention provides an antiperspirant/deodorant composition comprising: a) 2 w/w %-30 w/w % of an antiperspirant/deodorant active; (b) 0.01 w/w %-20 w/w % of an emulsifier, wherein the emulsifier contains one or more polyglycerol esters having an HLB value between 3 and 16, and (c) 30 w/w %-95 w/w % of an aqueous solvent, based on the total weight of the composition; and wherein the composition is an oil-in water emulsion for an aerosol, pump spray, liquid, roll-on, lotion, cream, gel, stick, or deodorant wipes.

The following examples illustrate the invention without limitation. All parts and percentages are given by weight unless otherwise indicated.

It will be understood that each of the elements described in the examples below, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims.

The foregoing embodiments are presented by way of example only; the scope of the present disclosure is to be limited only by the following claims.

### Example 1

TABLE 1
Antiperspirant/deodorant composition 1A

| Ingredients | w/w % conc. |
|---|---|
| Aluminum chlorohydrate (50% solution in water) | 10.00 |
| polyglyceryl-10 stearate | 5.00 |
| water | 85.00 |

Composition 1A was prepared by adding the aqueous phase ingredients to a main vessel at room temperature; then slowly increasing the temperature of the aqueous phase up to 60° C., whilst stirring with an overhead stirrer. The aqueous phase is stirred at 60° C. until complete dissolution, after which the reaction is cooled to 50° C., at which time the emulsion is sheared for 3 min at 3000 RPM.

### Example 2

TABLE 2
Antiperspirant/deodorant composition 2A

| | w/w % conc. |
|---|---|
| Ingredients phase A | |
| water | 71.00 |
| polyglyceryl-10 stearate | 5.00 |
| glycerol | 4.00 |
| Ingredients phase B | |
| Aluminum chlorohydrate (50% solution in water) | 20.00 |

Composition 2A was prepared by first preparing phase A then adding phase B to the mixture. Phase A is prepared by adding the phase A ingredients to a main vessel at room temperature; then slowly increasing the temperature of the aqueous phase A up to 60° C., whilst stirring with an overhead stirrer. The aqueous phase A is stirred at 60° C. until complete dissolution, after which the reaction is cooled to 40° C., at which time the phase B ingredient is added and the emulsion is sheared for 2 min at 4000 RPM at 40° C.

Stability testing for Composition 2A was performed at about 25° C. to about 45° C. respectively, for 1 month; with results showing complete stability of the composition after the 1 month period.

### Example 3. Roll-on Formulation

TABLE 3
Formulation for roll-on deodorant containing oil.

| Raw Material | w/w % | Range Levels |
|---|---|---|
| Water | qs | |
| Polyglyceryl-10 Dipalmitate | 4.00 | 3-8 |
| Aluminium Chlorohydrate (50% | 20.00 | 10-40 |
| solution in water) | | |
| Sunflower Oil | 3.00 | 0-5 |
| Polyglycerin-10 | 3.00 | 0-5 |
| Gluconolactone, sodium benzoate & | 1.00 | 0.5-1.5 |
| calcium gluconate | | |
| Fragrance | 1.00 | 0-2 |

Formulation in Table 3 provides a roll on deodorant/antiperspirant containing an oil. The formulation is prepared by adding water/aqua to a main vessel and heating to 50° C.-85° C., while stirring at a speed of 100-800 rpm. The oil/emollient; humectant and/or preservative (if required) is added to the main vessel then stirred until dissolution. The aluminum chlorohydrate is added to the main vessel. Separately heat the polyglyceryl ester to 50° C.-85° C. then add to the main vessel. Once added, continue to stir the formulation for 20-120 minutes or until full dissolution of the polyglyceryl ester. Cool to <50° C. and add fragrance (if required). Transfer vessel to a Silverson mixer and shear the product for 3 minutes at 3000 rpm at a temperature of 30° C.-50° C. (avoiding aeration of the product).

### Example 4. Roll-on Formulation

TABLE 4
Formulation for a roll-on antiperspirant/deodorant without oil.

| Raw Material | w/w % | w/w % |
|---|---|---|
| Water | qs | qs |
| Polyglyceryl-10 Dipalmitate | 4.00 | 6.00 |
| Aluminium Chlorohydrate (50% solution in water) | 20.00 | 20.00 |
| Polyglycerin-10 | 4.00 | 4.00 |
| Gluconolactone, sodium benzoate & calcium gluconate | 1.50 | — |

Formulation in Table 4 provides a roll-on antiperspirant/deodorant without oil; whereby 4A contains a preservative and 4B does not. Sample is prepared by adding water and heat to main the vessel to 50° C.-85° C.; then begin to stir with an overhead stirrer at a speed of 100-800 rpm. The preservative and humectant (if required) is then added into the main vessel and stir until dissolution. The aluminum chlorohydrate is then added into the main vessel. Separately heat the polyglyceryl ester to 50° C.-85° C. then added to the main vessel. The entire mixture is stirred for 20-120 minutes or until full dissolution of the polyglyceryl ester. Cool to <50° C. and add fragrance (if required). Transfer vessel to a Silverson mixer and shear the product for 3 minutes at 3000 rpm at a temperature of 30-50° C. (avoiding aeration of the product).

### Example 5. Roll-on Antiperspirant

TABLE 5
Oil-in-water emulsion for roll-on antiperspirant.

| Raw Material | w/w % |
|---|---|
| Aqueous phase | |
| Water | qs |
| Polyglyceryl-10 oleate | 0.60 |
| Aluminium Chlorohydrate (50% solution in water) | 20.00 |
| Glycerin | 4.00 |
| Oil phase | |
| Helianthus annuus (sunflower seed oil) | 4.00 |
| Polyglyceryl-10-dipalmitate or Polyglyceryl distearate | 2.60 |

Formulation in Table 5 provides a roll-on antiperspirant/deodorant wherein the composition is an oil-in-water emulsion. Formulation is prepared by adding water/aqua to a main vessel and heating to 50° C.-85° C. and stirring with an overhead stirrer at a speed of 100-800 rpm. Then add the remaining aqueous phase (Phase A) ingredients into main vessel, Separately heat the oil phase (Phase B) 50° C.-85° C., then add to the main vessel. At the required temperature, add the oil phase into the main vessel and slowly mix with the water phase (Phase A). Keep stirring at temperature for 10 mins or until full dissolution. Cool to <50° C. and add fragrance (if required). Transfer vessel to a Silverson mixer and shear the product for 3 minutes at 3000 rpm at a temperature of 30° C.-50° C. (avoiding aeration of the product).

### Example 6. Formulation for Various Antiperspirants

TABLE 6
Oil-free and humectant-free structured water systems.

| | w/w % | w/w % |
|---|---|---|
| Raw Material | #6A | #6B |
| Water | qs | qs |
| Polyglyceryl-10 dipalmitate | 4.00 | 6.50 |
| Aluminium Chlorohydrate (50% | — | 20.00 |
| solution in water) | | |
| fragrance | 0.80 | 1.00 |
| Gluconolactone, sodium benzoate & | 1.00 | 1.00 |
| calcium gluconate | | |
| Polyglycerin-10 | 3.00 | 3.00 |

Formulation in Table 6 provide a roll-on or cream antiperspirant/deodorant whereby the composition is an oil-free structured water system. Formulations in Table 6 are prepared as described in example 4.

### Example 7. Wipe or Lotion Antiperspirant

TABLE 7
Oil-in-water emulsion for wipe or lotion antiperspirant.

| Raw Material | w/w % |
|---|---|
| Aqueous phase | |
| Water | qs |
| Polyglyceryl-10 stearate | 0.60 |
| Aluminium Chlorohydrate (50% solution in water) | 20.00 |
| Glycerin | 4.00 |
| Oil phase | |
| Helianthus annuus (sunflower seed oil) | 4.00 |
| Polyglyceryl-10-laurate | 2.60 |

Formulation in Table 7 provides a composition for wipe or lotion antiperspirant/deodorant wherein the composition is an oil-in-water emulsion. Formulation in Table 7 is prepared as described in example 5.

### Example 8. Roll on or Cream Antiperspirant

TABLE 8
Oil-in-water emulsion for roll-on or cream antiperspirant,

| Raw Material | w/w % |
|---|---|
| Aqueous phase | |
| Water | qs |
| Polyglyceryl-10 oleate | 2.60 |
| Aluminium Chlorohydrate (50% solution in water) | 20.00 |
| Glycerin | 4.00 |
| Oil phase | |
| Helianthus annuus (sunflower seed oil) | 4.00 |
| Polyglyceryl-10-dipalmitate | 0.60-2.60 |

Formulation in Table 8 provides a roll-on or cream antiperspirant/deodorant whereby the composition is an oil-in-water emulsion. The addition of the polyglyceryl-10-dipalmitate can vary in the composition provided to adjust viscosity accordingly, for the desired result. Formulation in Table 8 is prepared as described in example 5.

### Example 9. Roll-on or Cream Antiperspirant

TABLE 9
Oil-in-water emulsion for roll-on or cream antiperspirant.

| Raw Material | w/w % |
|---|---|
| Aqueous phase | |
| Water | qs |
| Polyglyceryl-10 oleate | 0.60 |
| Polyglyceryl-10 dipalmitate | 0.60 |
| Aluminium Chlorohydrate (50% solution in water) | 20.00 |
| Glycerin | 4.00 |
| Oil phase | |
| Helianthus annuus (sunflower seed oil) | 4.00 |
| Polyglyceryl-6-distearate | 2.60 |

Formulation in Table 9 provides a roll-on or cream antiperspirant/deodorant wherein the composition is an oil-in-water emulsion containing a 3 polyglyceryl ester system. Formulation in Table 9 is prepared as described in example 5.

### Example 10. Roll-on or Cream Antiperspirant

TABLE 10
Oil-in-water emulsion for roll-on or cream antiperspirant.

| Raw Material | w/w % |
|---|---|
| Aqueous phase | |
| Water | qs |
| Polyglyceryl-10 oleate | 3.60 |
| Polyglyceryl-10 Dipalmitate | 3.60 |
| Aluminium Chlorohydrate (50% solution in water) | 20.00 |
| Oil phase | |
| Helianthus annuus (sunflower seed oil) | 4.00 |

Formulation in Table 10 provides a roll-on or cream antiperspirant or deodorant whereby the composition is an oil-in-water emulsion. Formulation in Table 10 is prepared as described in example 5.

### Example 11 Formulations for Various Antiperspirants

TABLE 11
Oil-free and humectant-free structured water systems.

| | w/w % | w/w % | w/w % | w/w % | w/w % |
|---|---|---|---|---|---|
| Raw Material | #11A | #11B | #11C | #11D | #11E |
| Water | qs | qs | qs | qs | qs |
| Polyglyceryl-10 stearate | 4-6.00 | 6-10.00 | 5.00 | 5.00 | 3.50 |
| or polyglyceryl-10 | | | | | |
| palmitate | | | | | |
| Aluminium | 20.00 | 20.00 | 20.00 | — | 20.00 |
| Chlorohydrate (50% | | | | | |
| solution in water) | | | | | |
| Glycerin | 4.00 | 4.00 | 4.00 | — | — |
| Gluconolactone, sodium | — | — | — | — | 1.5 |
| benzoate & calcium | | | | | |
| gluconate | | | | | |

Formulation #11A focused on a roll-on or cream antiperspirant whereby the composition is an oil-free and humectant-free structured water system; whereas Formulation #11C focused on roll-on or cream antiperspirant which is an oil-free structured water system containing humectant(s); additionally Formulation 11E contains the same structured water system however the composition contains a preservative(s). Formulation #11B and Formulation #11D focused on soft-solid or stick antiperspirant/deodorant which is oil free and humectant free structured water system; however Formulation 11D does not contain an antiperspirant active. Formulations in Table 11 are prepared as described in example 4.

### Example 12. Oil-in-Water Emulsion Antiperspirant

TABLE 12
Oil-in-water emulsion for roll-on or cream antiperspirant

| Raw Material | w/w % |
|---|---|
| Water | 66.50 |
| Polyglyceryl-10 dipalmitate | 4.50 |
| Aluminium Chlorohydrate (50% solution in water) | 20.00 |
| Gluconolactone, sodium benzoate & calcium gluconate | 1.00 |
| Fragrance | 1.00 |
| Helianthus annuus (sunflower seed oil) | 4.00 |
| Polyglycerin-10 | 3.00 |

The roll-on or cream antiperspirant/deodorant composition is an oil-in-water emulsion whereby the oil can be substituted for any oil such as olive, canola, sunflower, safflower, hemp seed oil, marula oil, argan oil, and the like. Formulation in Table 12 is prepared as described in example 3.

### Example 13. Oil-in-Water Emulsion for Roll-on or Cream Antiperspirant

TABLE 13
Formulation with preservative.

| | w/w % | w/w % | w/w % |
|---|---|---|---|
| Raw Material | #13A | #13B | #13C |
| Phase A | | | |
| Water | qs | qs | qs |
| Disodium EDTA | 0.10 | 0.10 | 0.10 |
| Phase B | | | |
| Polyglyceryl-10 oleate | 3.60 | 3.60 | — |
| Polyglyceryl-10 stearate | 2.40 | 2.40 | 3.00 |
| Polyglyceryl-6-caprylate/caprate | — | — | 3.00 |
| Tocopherol | 0.10 | 0.10 | 0.10 |
| Butylated hydroxytoluene (BHT) | 0.10 | 0.10 | 0.10 |
| Grape seed oil | — | 3.00 | 3.00 |
| Dimethicone | — | 1.00 | 1.00 |
| Phase C | | | |
| Aluminium Chlorohydrate (50% | 20.00 | 40.00 | 40.00 |
| solution in water) | | | |
| Phase D | | | |
| DMDMH | 0.30 | 0.30 | 0.30 |

DMDMH: (1,3-Bis(hydroxymethyl)-5,5-dimethylimidazolidine-2,4-dione)

Formulation #13A and 13B focused on roll-on or cream antiperspirant/deodorant wherein the composition is an oil-in-water emulsion. Formulation #13C focused on lotion or wipe antiperspirant/deodorant, wherein the composition is also an oil-in-water emulsion.

Formulation in Table 13 is prepared by separately mixing the ingredients of Phase A and Phase B together at room temperature. Each phase is heated individually in separate vessels until they reach 50° C.-85° C. Phase B is added to Phase A whilst stirring at 500-2000 rpm using overhead stirrer. Sample is stirred at temperature for 10 mins or until full dissolution. Whilst stirring allow the solution to cool to <60° C. then slowly add Phase C. Continue mixing with the same speed until temperature drops below 40° C., then add Phase D and mix until completely blended.

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. An antiperspirant/deodorant composition comprising:
(a) 2 w/w %-30 w/w % of an antiperspirant/deodorant active;
(b) 0.01 w/w %-20 w/w % of an emulsifier, wherein the emulsifier contains one or more polyglycerol esters having an HLB value between 3-16, and
(c) 30 w/w %-95 w/w % of an aqueous solvent, based on the total weight of the composition.

2. The antiperspirant/deodorant composition according to claim 1, wherein (a) the antiperspirant/deodorant active is present in an amount of 5 w/w %-20 w/w %; (b) the emulsifier is present in an amount of 1 w/w %-8 w/w %; and c) an aqueous solvent present in an amount of 60 w/w %-95 w/w %, based on the total weight of the composition.

3. The antiperspirant/deodorant composition according to claim 1, wherein the antiperspirant/deodorant active comprises an aluminum salt, a zinc salt, a zirconium salt, a deodorant active, or mixtures thereof.

4. The antiperspirant/deodorant composition according to claim 2, wherein the antiperspirant/deodorant active comprises an aluminum salt, a zinc salt, a zirconium salt, a deodorant active, or mixtures thereof

5. The antiperspirant/deodorant composition according to claim 3, wherein the antiperspirant/deodorant active comprises aluminum chlorohydrate.

6. The antiperspirant/deodorant composition according to claim 4, wherein the antiperspirant/deodorant active comprises aluminum chlorohydrate

7. The antiperspirant/deodorant composition according to claim 1, wherein the emulsifier comprises a polyglycerol ester, having an HLB value between 3-16, derived from (a) a polyglycerol component built up from 2 molecules to 12 molecules of glycerol, based on an average, and (b) a fatty acid comprising caprylic acid, capric acid, lauric acid, myristic acid, palmitic acid, stearic acid, arachidic acid, behenic acid, lignoceric acid, cerotic acid, oleic acid, decaoleic acid or mixtures thereof.

8. The antiperspirant/deodorant composition according to claim 7, wherein the emulsifier comprises one or more of a polyglycerol ester, having an HLB value between 3-16, of polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, polyglyceryl-10-palmitate, polyglyceryl-10-laurate, polyglyceryl-10 caprylate/caprate.

9. The antiperspirant/deodorant composition according to claim 1, wherein the aqueous solvent comprises water, aqueous alcohols, ammonia water, acid solutions, salt solutions, water-miscible organic solvents or mixtures thereof.

10. The antiperspirant/deodorant composition according to claim 9, wherein the aqueous solvent comprises water.

11. The antiperspirant/deodorant composition according to claim 1, further comprising an emollient, wherein the emollient is present in an amount of 1 w/w %-10 w/w %, based on the total weight of the composition.

12. The antiperspirant/deodorant composition according to claim 1, further comprising an emollient, wherein the emollient is present in an amount less than 1%.

13. The antiperspirant/deodorant composition according to claim 1, wherein the composition is free of an emollient.

14. The antiperspirant/deodorant composition according to claim 1, further comprising an additional emulsifier, wherein the additional emulsifier comprises one or more of an ethoxylated fatty alcohols and ethoxylated fatty acids, ethoxylated sorbitan esters, in particular fatty esters of ethoxylated sorbitan.

15. The antiperspirant/deodorant composition according to claim 14, wherein the additional emulsifier comprises an ethoxylated fatty alcohol.

16. The antiperspirant/deodorant composition according to claim 14, wherein the polyglycerol ester to the additional emulsifier are present in a weight ratio of about 1:10 to 10:1.

17. The antiperspirant/deodorant composition according to claim 1, further comprising a humectant, wherein the humectant is present in an amount of about 0.01 w/w %-10 w/w %, based on the total weight of the composition.

18. The antiperspirant/deodorant composition according to claim 17, wherein the humectant comprises a polyol.

19. The antiperspirant/deodorant composition according to claim 18, wherein the humectant comprises glycerol.

20. The antiperspirant/deodorant composition according to claim 1, wherein the composition comprises an oil-in-water emulsion.

21. The antiperspirant/deodorant composition according to claim 1, further comprising a viscosity modifier, fragrance, co-surfactant, cosmetic additive, biocidal agent, deodorant active, chelating agent, antioxidant, essential oil, preservative, vitamin, moisturizer, or mixtures thereof.

22. The antiperspirant/deodorant composition according to claim 1, further comprising a preservative, wherein the preservative comprises a blend of gluconolactone, sodium benzoate and calcium gluconate.

23. The antiperspirant/deodorant composition according to claim 22, wherein the preservative is present in an amount of 0.01 w/w %-10 w/w %, based on the total weight of the composition.

24. The antiperspirant/deodorant composition according to claim 1, in the form of an aerosol, pump spray, liquid, roll-on, lotion, cream, gel, stick, or deodorant wipes.

25. An antiperspirant/deodorant composition comprising:
a) 2 w/w %-30 w/w % of an antiperspirant/deodorant active;
(b) 0.01 w/w %-20 w/w % of an emulsifier, wherein the emulsifier contains one or more polyglycerol esters having an HLB value between 3 and 16, and
(c) 30 w/w %-95 w/w % of an aqueous solvent, based on the total weight of the composition; and
wherein the composition is an oil-in water emulsion for an aerosol, pump spray, liquid, roll-on, lotion, cream, gel, stick, or deodorant wipe.
